(19) United States
(12) Patent Application Publication
(10) Pub. No.: US 2024/0058242 A1
(43) Pub. Date: Feb. 22, 2024

(54) SKIN OINTMENT AND PREPARATION METHOD THEREFOR

(71) Applicant: The 373 Company LLC, Bismarck, ND (US)

(72) Inventor: Randall E. Drown, Bismarck, ND (US)

(21) Appl. No.: 18/450,192

(22) Filed: Aug. 15, 2023

Related U.S. Application Data
(60) Provisional application No. 63/371,576, filed on Aug. 16, 2022.

(51) Int. Cl. A61K 8/36; A61K 8/34; A61K 8/19; A61Q 19/00

(52) U.S. Cl. CPC A61K 8/361; A61K 8/342; A61K 8/19; A61Q 19/007; A61K 2800/805; A61K 2800/5922

(57) ABSTRACT

A process for the manufacture of a skin ointment includes creating a eutectic mixture that includes an oleic acid; and adding water that is preheated to 178 degrees F. directly into the eutectic mixture at 178 degrees F. A skin ointment that includes 10-14% oleic acid; 7-11% palmitic acid; 7-11% stearic acid; 6-10% cetearyl alcohol; 1-3% boric acid 99%+; 1-2% bismuth sub carbonate 99%+; and the remaining part being purified water. A 2 oz skin ointment portion that includes 3.18 g oleic acid; 2.47 g palmitic acid; 2.47 g stearic acid; 2.18 g cetearyl alcohol; 0.61 g boric acid 99%+; 0.32 g bismuth sub carbonate 99%+; and 16.11 g Purified Water.

### CROSS REFERENCE TO RELATED APPLICATIONS

The present disclosure claims priority to United States Provisional Patent Application Number Ser. No. 63/371,576 filed Aug. 16, 2022 (01656-DRO).

### BACKGROUND

The present disclosure relates to a skin ointment, preparation method and application thereof.

Various prescriptions and steroids are available to treat skin conditions. Disadvantageously, such prescriptions and steroids necessarily require a doctor's involvement and may have side-effects.

### SUMMARY

A process for the manufacture of a skin ointment according to one disclosed non-limiting embodiment of the present disclosure includes creating a eutectic mixture that includes an oleic acid; and adding water that is preheated to 178 degrees F. directly into the eutectic mixture at 178 degrees F.

A further embodiment of any of the foregoing embodiments of the present disclosure includes, wherein creating a eutectic mixture that includes an oleic acid further includes: using a bain-marie heated bath, bring transfer temperature to 178 degrees F.; adding oleic acid to bain-marie and heat to 178 degrees F.; and adding palmitic acid while stirring.

A further embodiment of any of the foregoing embodiments of the present disclosure includes, wherein creating a eutectic mixture that includes an oleic acid further includes: adding boric acid while stirring.

A further embodiment of any of the foregoing embodiments of the present disclosure includes, wherein creating a eutectic mixture that includes an oleic acid further includes: with cavitation present, stir briskly.

A further embodiment of any of the foregoing embodiments of the present disclosure includes, wherein creating a eutectic mixture that includes an oleic acid further includes: adding bismuth sub carbonate, then continue stirring to form a completed eutectic mixture.

A further embodiment of any of the foregoing embodiments of the present disclosure includes, wherein adding water that is preheated to 178 degrees F. directly into the eutectic mixture at 178 degrees F. further includes: begin emulsification process by submersing an electric emulsifying tool into mixture for 30 seconds; and let mixture rest and cool for 4 minutes.

A further embodiment of any of the foregoing embodiments of the present disclosure includes, wherein adding water that is preheated to 178 degrees F. directly into the eutectic mixture at 178 degrees F. further includes: observe and separation of water and eutectic mixture; and blend again as per (a) on high speed for 30 seconds.

A further embodiment of any of the foregoing embodiments of the present disclosure includes wherein adding water that is preheated to 178 degrees F. directly into the eutectic mixture at 178 degrees F. further includes: let mixture rest and cool for 4 minutes; and observe mixture for any separation of eutectic mixture and water.

A further embodiment of any of the foregoing embodiments of the present disclosure includes, wherein creating a eutectic mixture that includes an oleic acid further includes: using a bain-marie heated bath, bring transfer temperature to 178 degrees F.; adding oleic acid to bain-marie and heat to 178 degrees F.; adding palmitic acid while stirring; stir occasionally until completely melted; return to 178 degrees F.; adding stearic acid while stirring; stir occasionally until completely melted; return to 178 degrees F.; adding boric acid while briskly and continuously stirring and continue stirring for one minute; with cavitation present, stir briskly while continuing to step “k”; and while stirring, adding bismuth sub carbonate, then continue stirring for one minute to form a completed eutectic mixture.

A further embodiment of any of the foregoing embodiments of the present disclosure includes, wherein adding water that is preheated to 178 degrees F. directly into the eutectic mixture at 178 degrees F. further includes: begin emulsification process by submersing an electric emulsifying tool into mixture for 30 seconds; let mixture rest and cool for 4 minutes; observe and separation of water and eutectic mixture; blend again as per (a) on high speed for 30 seconds; let mixture rest and cool for 4 minutes; and observe mixture for any separation of eutectic mixture and water.

A skin ointment according to one disclosed non-limiting embodiment of the present disclosure includes 10-14% oleic acid; 7-11% palmitic acid; 7-11% stearic acid; 6-10% cetearyl alcohol; 1-3% boric acid 99%+; 1-2% bismuth sub carbonate 99%+; and the remaining part being purified water.

A further embodiment of any of the foregoing embodiments of the present disclosure includes 12% oleic acid.

A further embodiment of any of the foregoing embodiments of the present disclosure includes 9% palmitic acid.

A further embodiment of any of the foregoing embodiments of the present disclosure includes 9% stearic acid.

A further embodiment of any of the foregoing embodiments of the present disclosure includes 8% cetearyl alcohol.

A further embodiment of any of the foregoing embodiments of the present disclosure includes 2% boric acid 99%+.

A further embodiment of any of the foregoing embodiments of the present disclosure includes 1% bismuth sub carbonate 99%+.

A further embodiment of any of the foregoing embodiments of the present disclosure includes, wherein the batch produces 220, 2 oz portions.

A further embodiment of any of the foregoing embodiments of the present disclosure includes 12% oleic acid; 9% palmitic acid; 9% stearic acid; 8% cetearyl alcohol; 2% boric acid 99%+; 1% bismuth sub carbonate 99%+; and the remaining part being purified water.

A 2 oz skin ointment portion, including the following components by weight according to one disclosed non-limiting embodiment of the present disclosure includes: 3.18 g oleic acid; 2.47 g palmitic acid; 2.47 g stearic acid; 2.18 g cetearyl alcohol; 0.61 g boric acid 99%+; 0.32 g bismuth sub carbonate 99%+; and 16.11 g Purified Water.

The foregoing features and elements may be combined in various combinations without exclusivity, unless expressly indicated otherwise. These features and elements as well as the operation thereof will become more apparent in light of the following description and the accompanying drawings. It should be appreciated that however the following description and drawings are intended to be exemplary in nature and non-limiting.

### DETAILED DESCRIPTION

A skin ointment, in one disclosed non-limiting embodiment, although primarily disclosed for treatment of eczema, also soothes damaged skin, rehydrate dry skin and provides comfort to a user.

One formula for the skin ointment, in one disclosed non-limiting embodiment may include, by weight percentages: 10-14% Oleic Acid; 7-11% Palmitic Acid; 7-11% Stearic Acid; 6-10% Cetearyl Alcohol; 1-3% Boric Acid 99%+; 1-2% Bismuth Sub carbonate 99%+; and the remaining part being Purified Water.

One formula for the skin ointment, in one disclosed non-limiting embodiment may include, by weight percentages: 12% Oleic Acid; 9% Palmitic Acid; 9% Stearic Acid; 8% Cetearyl Alcohol; 2% Boric Acid 99%+; 1% Bismuth Sub carbonate 99%+; and the remaining part being Purified Water.

More specifically, one example formula for the skin ointment, in one disclosed non-limiting embodiment may include, by weight, the following components:

| | | portion | batch | |
|---|---|---|---|---|
| 1. | Oleic Acid | 3.18181818 g | 1400 | g |
| 2. | Palmitic Acid | 2.47159091 g | 1087.5 | g |
| 3. | Stearic Acid | 2.47159091 g | 1087.5 | g |
| 4. | Cetearyl Alcohol | 2.18181818 g | 960 | g |
| 5. | Boric Acid 99%+ | 0.61363636 g | 270 | g |
| 6. | Bismuth Sub carbonate 99%+ | 0.32386364 g | 142.5 | g |
| 7. | Purified Water | 16.11 g | 1.95313 | gallon |
| | | (0.56818304 oz.) | | |

A batch using the example formula may produce 220, 2 oz portions.

A process for the manufacture of the skin ointment, in one disclosed non-limiting embodiment includes:

Step 1. Create a eutectic mixture:
a. Using a Bain-Marie heated bath, bring transfer temperature to 178 degrees F.
b. Adding Oleic Acid to Bain-Marie. Heat Oleic Acid to 178 degrees F.
c. Adding Palmitic Acid while stirring.
d. Stir occasionally until completely melted.
e. Return to 178 degrees F.
f. Adding Stearic Acid while stirring.
g. Stir occasionally until completely melted.
h. Return to 178 degrees F.
i. Adding Boric Acid while briskly and continuously stirring-continue brisk stirring for one minute.
j. Stop stirring and closely observe eutectic mixture for noticeable cavitation and if present continue. If cavitation not present, dump batch and go back to step 1. With cavitation present, stir briskly while continuing to Step “k” below.
k. While briskly stirring, adding Bismuth Sub carbonate. Continue stirring for one minute to form a Completed Eutectic Mixture. A eutectic mixture is a specific composition of at least two solid components that produces a change of phase to liquid at a certain temperature. This temperature receives the name of eutectic point temperature and corresponds to the minimum melting temperature of the different possible compositions.

Step 2. Adding water (pre heated to 178 degrees F.) directly into eutectic mixture at 178 degrees F.
a. Immediately begin emulsification process by submersing an electric emulsifying tool into mixture. Emulsify on high speed. Blend mixture for 30 seconds.
b. Let mixture rest and cool for 4 minutes.
c. Observe and separation of water and eutectic mixture.
d. Blend again as per (a) on high speed for 30 seconds.
e. Let mixture rest and cool for 4 minutes.
f. Observe mixture for any separation of eutectic mixture and water.

If no visible separation exists, communicate completed formula to packaging line. If separation exits, blend for 30 seconds on low speed, then communicate to packaging line. During the filling process, continually observe mixture for separation in tank. If separation present, turn off tank mixer and emulsify 30 seconds on low speed. Continue filling, repeating Step 2f above as needed.

The packaging line requirements includes a heated mixer tank capable of maintaining fill temperature of 120 plus degrees F. and an emulsifying wand.

Applicant has found that the skin ointment treats eczema and generally dry, cracked skin. Additionally, some users have found that the skin ointment can ease minor sunburns and other types of skin conditions. The skin ointment may also be used for hemorrhoids, cold sores, diaper rash, sunburns, burns, chapped lips, yeast infection, bug bites, Poison Ivy, oak rashes, and soothes, repairs and softens skin.

Although the different non-limiting embodiments have specific illustrated components, the embodiments of this invention are not limited to those particular combinations. It is possible to use some of the components or features from any of the non-limiting embodiments in combination with features or components from any of the other non-limiting embodiments.

The foregoing description is exemplary rather than defined by the limitations within. Various non-limiting embodiments are disclosed herein, however, one of ordinary skill in the art would recognize that various modifications and variations in light of the above teachings will fall within the scope of the appended claims. It is therefore to be appreciated that within the scope of the appended claims, the disclosure may be practiced other than as specifically described. For that reason, the appended claims should be studied to determine true scope and content.

What is claimed is:

1. A process for the manufacture of a skin ointment, comprising:
creating a eutectic mixture that comprises an oleic acid; and
adding water that is preheated to 178 degrees F. directly into the eutectic mixture at 178 degrees F.

2. The process as recited in claim 1, wherein creating a eutectic mixture that comprises an oleic acid further comprises:
using a bain-marie heated bath, bring transfer temperature to 178 degrees F.;
adding oleic acid to bain-marie and heat to 178 degrees F.; and
adding palmitic acid while stirring.

3. The process as recited in claim 2, wherein creating a eutectic mixture that comprises an oleic acid further comprises:
adding boric acid while stirring.

4. The process as recited in claim 3, wherein creating a eutectic mixture that comprises an oleic acid further comprises:
with cavitation present, stir briskly.

5. The process as recited in claim 4, wherein creating a eutectic mixture that comprises an oleic acid further comprises:
adding bismuth sub carbonate, then continue stirring to form a completed eutectic mixture.

6. The process as recited in claim 5, wherein adding water that is preheated to 178 degrees F. directly into the eutectic mixture at 178 degrees F. further comprises:
a. begin emulsification process by submersing an electric emulsifying tool into mixture for 30 seconds; and
b. let mixture rest and cool for 4 minutes.

7. The process as recited in claim 6, wherein adding water that is preheated to 178 degrees F. directly into the eutectic mixture at 178 degrees F. further comprises:
c. observe and separation of water and eutectic mixture; and
d. blend again as per (a) on high speed for 30 seconds.

8. The process as recited in claim 6, wherein adding water that is preheated to 178 degrees F. directly into the eutectic mixture at 178 degrees F. further comprises:
e. let mixture rest and cool for 4 minutes; and
f. observe mixture for any separation of eutectic mixture and water.

9. The process as recited in claim 1, wherein creating a eutectic mixture that comprises an oleic acid further comprises:
a. using a bain-marie heated bath, bring transfer temperature to 178 degrees F.;
b. adding oleic acid to bain-marie and heat to 178 degrees F.;
c. adding palmitic acid while stirring;
d. stir occasionally until completely melted;
e. return to 178 degrees F.;
f. adding stearic acid while stirring;
g. stir occasionally until completely melted;
h. return to 178 degrees F.;
i. adding boric acid while briskly and continuously stirring and continue stirring for one minute;
j. with cavitation present, stir briskly while continuing to step “k”; and
k. while stirring, adding bismuth sub carbonate, then continue stirring for one minute to form a completed eutectic mixture.

10. The process as recited in claim 9, wherein adding water that is preheated to 178 degrees F. directly into the eutectic mixture at 178 degrees F. further comprises:
a. begin emulsification process by submersing an electric emulsifying tool into mixture for 30 seconds;
b. let mixture rest and cool for 4 minutes;
c. observe and separation of water and eutectic mixture;
d. blend again as per (a) on high speed for 30 seconds;
e. let mixture rest and cool for 4 minutes; and
f. observe mixture for any separation of eutectic mixture and water.

11. A skin ointment, comprising the following components by weight percentage:
10-14% oleic acid;
7-11% palmitic acid;
7-11% stearic acid;
6-10% cetearyl alcohol;
1-3% boric acid 99%+;
1-2% bismuth sub carbonate 99%+; and
the remaining part being purified water.

12. The skin ointment as recited in claim 11, further comprising:
12% oleic acid.

13. The skin ointment as recited in claim 11, further comprising:
9% palmitic acid.

14. The skin ointment as recited in claim 11, further comprising:
9% stearic acid.

15. The skin ointment as recited in claim 11, further comprising:
8% cetearyl alcohol.

16. The skin ointment as recited in claim 11, further comprising:
2% boric acid 99%+.

17. The skin ointment as recited in claim 11, further comprising:
1% bismuth sub carbonate 99%+.

18. The skin ointment as recited in claim 11, wherein the batch produces 220, 2 oz portions.

19. The skin ointment as recited in claim 11, further comprising:
12% oleic acid;
9% palmitic acid;
9% stearic acid;
8% cetearyl alcohol;
2% boric acid 99%+;
1% bismuth sub carbonate 99%+; and
the remaining part being purified water.

20. A 2 oz skin ointment portion, comprising the following components by weight:
3.18 g oleic acid;
2.47 g palmitic acid;
2.47 g stearic acid;
2.18 g cetearyl alcohol;
0.61 g boric acid 99%+;
0.32 g bismuth sub carbonate 99%+; and
16.11 g Purified Water.
